(19) United States
(12) Patent Application Publication
(10) Pub. No.: US 2016/0228231 A1
(43) Pub. Date: Aug. 11, 2016

(54) PERMEABLE POUCH FOR TISSUE CONTAINMENT

(71) Applicant: AlloSource, Centennial, CO (US)

(72) Inventors: Matthew James Southard, Denver, CO (US); Marina Katelyn Bull, Highlands Ranch, CO (US); Adrian C. Samaniego, Parker, CO (US); Kenneth Blood, Littleton, CO (US); Reginald Stilwell, Parker, CO (US)

(21) Appl. No.: 15/017,474

(22) Filed: Feb. 5, 2016

Related U.S. Application Data
(60) Provisional application No. 62/113,088, filed on Feb. 6, 2015.

(51) Int. Cl. A61F 2/00; B65B 43/04; B65B 7/02; B65D 81/00; A61F 2/02

(52) U.S. Cl. CPC A61F 2/0095; B65D 81/00; A61F 2/02; B65B 7/02; B65B 43/04

(57) ABSTRACT

Described are permeable pouches for tissue containment and methods for their use and manufacture. Exemplary pouches include a permeable material formed to create a cavity into which tissue graft material can be placed. Tissue graft material may be enclosed within a sealed pouch or within an unsealed pouch.

### CROSS-REFERENCES TO RELATED APPLICATIONS

This application claims the benefit of priority to U.S. Provisional Application No. 62/113,088, filed Feb. 6, 2015, the entire content of which is hereby incorporated by reference for all purposes.

### BACKGROUND

Medical grafting procedures often involve the implantation of autogenous, allograft, or synthetic grafts into a patient to treat a particular condition or disease. Grafts may remain intact and replace the patient's natural tissue or may be reabsorbed and replaced with the patient's natural tissue upon healing.

Allograft and autogenous tissue are both derived from humans. Autogenous tissue is derived from a person's own tissue, while allograft tissue is harvested from a different person. Allograft tissue is often taken from cadavers where a person has donated their tissue so that it can be used for the medical treatment of others.

Medical graft tissue is packaged for use by medical providers. In some instances, tissue grafts may be manipulated prior to implantation into a patient. For example, tissue graft material may be washed to remove solution in which it is stored.

The permeable pouch and manufacture methods described herein provide means for facilitating use of tissue grafts.

### BRIEF SUMMARY

Disclosed herein are permeable pouches for tissue containment and methods for their use and manufacture. Exemplary pouches include a permeable material formed to create a cavity into which tissue graft material can be placed. Tissue graft material may be enclosed within a sealed pouch or within an unsealed pouch.

In one aspect, provided is a pouch for tissue containment that includes at least one piece of permeable material having a geometrical shape and a cavity enclosed by the permeable material, the permeable material comprising seams formed therein to delineate the cavity.

In another aspect, provided is a method of making a pouch for tissue containment, the method including the steps of (a) providing a permeable material; (b) folding the permeable material into a geometrical configuration by bringing at least one edge or surface of the permeable material into contact with at least one other edge or surface of the permeable material, wherein the three-dimensional configuration comprises an internal cavity formed therein; and (c) forming a seal between the at least one edge or surface of the permeable material and the at least one other edge or surface of the permeable material.

In another aspect, provided is a method of making a pouch for tissue containment, the method including the steps of (a) providing a plurality of pieces of permeable material; (b) positioning the pieces of permeable material into a geometrical configuration by bringing at least one edge or surface of a first piece of permeable material into contact with at least one other edge or surface of a second piece of permeable material, wherein the geometrical configuration comprises an internal cavity formed therein; and (c) forming a seal between the at least one edge or surface of the first permeable material and the at least one edge or surface of the second permeable material.

In another aspect, provided is a method of treating a patient having a tissue defect site in need of a tissue graft, the method comprising: (a) providing a pouch for tissue containment; (b) opening the pouch to expose the tissue contained therein by separating at least some of the seams in the permeable material; (c) extracting the tissue from the cavity of the pouch; and (d) implanting the tissue into the tissue defect site of the patient. The pouch for tissue containment may include at least one piece of permeable material having a geometrical shape and a cavity enclosed by the permeable material, the permeable material comprising seams formed therein to delineate the cavity.

In another aspect, provided is a method of treating a patient with a permeable pouch containing tissue graft material, the method comprising inserting the pouch into a wound site of a patient in need of a tissue graft.

In another aspect, provided is a method of treating a patient having a tissue defect site in need of a tissue graft, the method comprising: (a) providing an unsealed pouch containing tissue; (b) unfolding the permeable material of the unsealed pouch to expose the tissue; and (c) implanting the tissue into the tissue defect site of the patient.

The above described and many other features and attendant advantages of embodiments of the present invention will become apparent and further understood by reference to the following detailed description when considered in conjunction with the accompanying drawings.

### BRIEF DESCRIPTION OF THE DRAWINGS

FIG. 1A shows an exemplary piece of permeable material that is folded vertically along its midline and having seams introduced on the left and right side of the material perpendicular to the fold line to form an open pouch. Tissue is shown positioned with in the cavity of the open pouch formed by the folded edge and the seams. A final seam is introduced across the edge of the material parallel to the fold line to enclose the tissue within the cavity of the pouch.

FIG. 1B depicts a flowchart of a method of manufacturing a permeable pouch containing tissue.

FIG. 1C shows an image of an open pouch that does not contain any tissue. The open pouch is formed from two pieces of permeable material having seams introduced on the left edge and the bottom edge.

FIG. 1D shows an image of the pouch depicted in FIG. 1C that contains tissue. The pouch has been sealed closed with a seam across its top edge and right edge to a closed pouch.

FIGS. 2A-2D depict various configurations of permeable material and the shape of the permeable pouch that can be made therefrom. The pouches depicted have a relatively flat geometry.

FIG. 3 depicts a flowchart of a method of manufacturing a permeable pouch containing tissue.

FIG. 4A-4E depict various configurations of permeable material and the shape of the permeable pouch that can be made therefrom. The pouches depicted have three-dimensional shapes.

FIG. 5 depicts a flowchart of a method of treating a patient with a defect site. The method involves washing and removing tissue from within a sealed permeable pouch, and implating the tissue into the defect site of the patient.

FIG. 6 depicts a flowchart of a method of treating a patient with a defect site. The method involves washing a sealed permeable pouch containing tissue and implanting the tissue into the defect site of the patient.

FIG. 7A depicts an open pouch having a flower-like shape with a central circular portion and cuts (perforations) delineated in the permeable material radiating out from the circular portion to the peripheral edges of the material. The cuts facilitate flexibility of the material. The material along the line of the cuts may separated to form separated portions of the material radiating out from the circular portion (radial portions, e.g., like flower petals).

FIG. 7B depicts a cross section of the open pouch of FIG. 7A positioned perpendicular to the opening of a container (such as a vial), the open pouch having a piece of tissue positioned on it. The tissue is positioned at least in part on top of the central circular portion. As shown on the right, the open pouch may be positioned within the container so that the central circular portion rests on bottom of the container and the radial portions of the material are positioned along the sides of the container enclosing (encircling) the tissue. There may be some overlap of the radial portions with each other. One or more of the radial portions may be collapsed internally away from the sides of the container (not shown).

FIG. 7C depicts a method of manufacturing a tissue product packaged within an open permeable pouch. Tissue may be positioned on an open pouch, and the pouch positioned around the tissue (e.g., by folding) to enclose it. The pouch and tissue may then be transferred to a storage container or packaging.

### DETAILED DESCRIPTION

Embodiments of the present invention encompass permable pouches for containing tissue. Such pouches can used to contain tissue such as for a tissue graft. For example, the pouches can be used to contain and manipulate tissue comprising particulate tissue. In another example, the pouches may be used to contain and manipulate multiple pieces of tissue simultaneously. Once tissue is inserted, the pouches contain the tissue and may permit, for example, transfer of the tissue into a storage container or packaging, transfer of the tissue from a storage container or packaging to a work surface, and washing of the tissue, and may also provide a uniform surface for retaining the tissue prior to implantation. The pouches may be formed from one or more pieces of permeable material such as a mesh material.

In some aspects, the pouches may be sealed pouches formed by sealing together portions of a permeable material, or portions of multiple permeable material members (such as to form a seal or seam). The seal may be manufactured in a way that when average mechanical stress, handling, and manipulation of the pouch occur; the pouch does not open. However, with moderate stress, the pouch can be opened by separating the seams to expose the tissue. In separating the seams, the permeable material can be laid flat to provide a uniform surface to retain the tissue prior to implantation. In some aspects, sealed pouches containing tissue graft material may be implanted directly into a patient in need of a tissue graft without removing the tissue graft material from the sealed pouch. In some aspects, the pouches may be unsealed pouches formed by folding or collapsing together portions of a permeable material. Unsealed pouches can be used to contain, transfer, and manipulate tissue graft material in a similar manner to sealed pouches. Unsealed pouches may also be unfolded to provide a uniform surface to retain the tissue prior to implantation.

The described pouches are useful for storing and manipulating various types of tissue material. In particularly, the pouches may be used with tissue products prepared for use as tissue grafts. The tissue is enclosed within the permeable material of the pouches and can be readily manipulated, such as by washing, prior to implantation into a defect site of a subject. Tissue for insertion or enclosure within the pouches may range in source, size, texture, and consisteny. For example, tissue may be dervied from harvested adipose tissue, epidermal tissue, bone tissue, and/or cartilage tissue, amongst others. The tissue may be in particulate form, with particulates including uniform or nonuniform particulate sizes. Alternatively, the tissue may be multiple larger pieces of tissue. In some instances, the tissue may be saturated with a wetting agent such as, for example, sterile water, saline, a cryoprotective solution, a preservative, a nutrititive culture medium, a polymer solution, or a combination of two or more thereof. In other instances, the tissue may be hydrated, partially hydrated, or dehydrated in form.

### Sealed Pouches

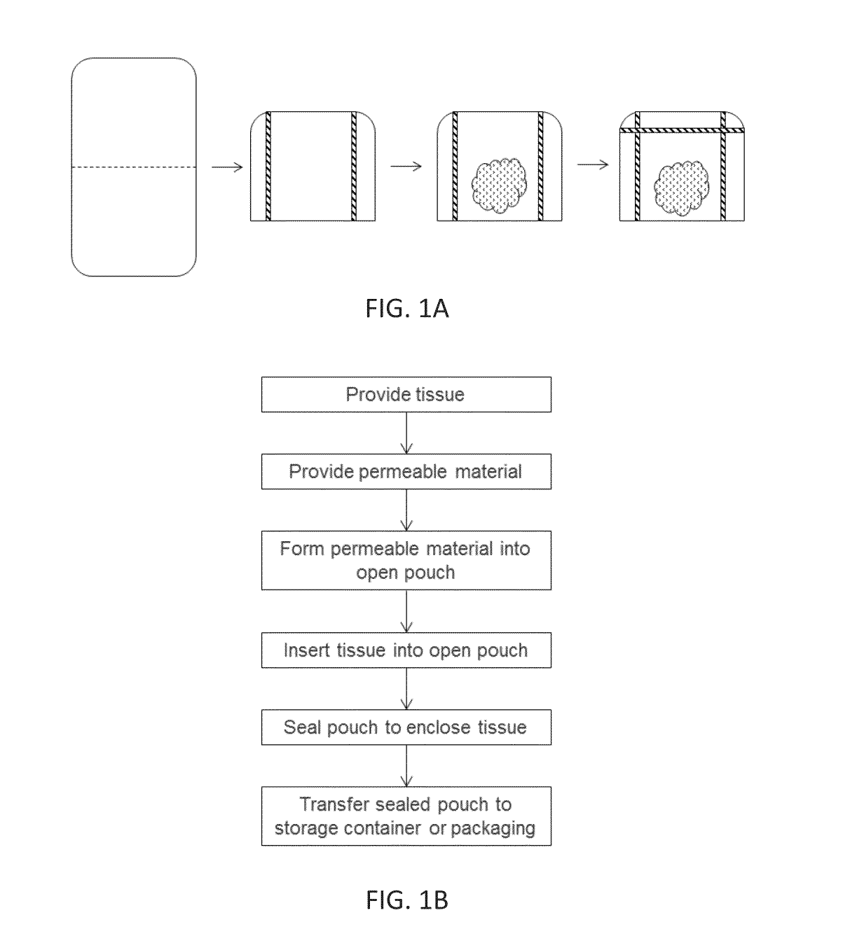

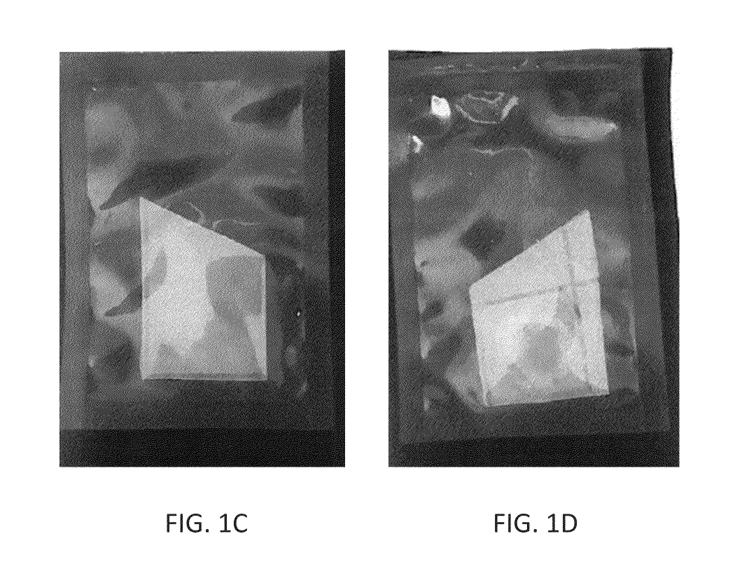

Turning now to the drawings, FIGS. 1A-1B depict the formation of an exemplary permeable pouch and its use to contain tissue. A permeable material is provided. The shape of the permeable material is selected so that, upon folding, the permeable material forms the desired geometry (geometric shape) for the pouch. In this example, the permeable material has a rectangular shape that is folded in half to form a bi-layer square shape. The position of the fold is indicated by the dashed line. By folding the permeable material, at least one edge or portion of the permeable material is brought into contact with at least one other edge or portion of the permeable material. A seal, or seam, may be formed to adjoin the edges or portions of the permeable material that are in contact with each other. In this example, two seams are formed in the permeable material (i.e. on each side of the bi-layer square shape perpendicular to the fold) forming a pouch with an opening defined between the layers of the permeable material opposite the folded edge thereof. Thus, a cavity (void) is formed within the open pouch. Tissue graft material may be inserted within the cavity via the opening of the pouch. Once the tissue is positioned within the cavity of the pouch, the opening(s) of the pouch may be sealed to fully enclose the tissue graft material within the permeable material. In this example, the opening in the pouch that is opposite the folded edge of the permeable material is sealed to enclose the tissue graft material. Thus, in some instances, the pouch may be first formed into an open pouch configuration into which the tissue is placed and then the pouch is sealed to fully enclose the tissue within the pouch. In other instances, the permeable material may be folded around the tissue graft material and then seams are formed in the permeable material to form and seal the pouch. In some instances, a sealed pouch may be placed into a non-permeable storage container or packaging such as, for example, one or more plastic or glass vials or bags. In some instances, the storage container or packaging may be filled with a wetting solution such as, for example, sterile water, saline, a cryoprotective solution, a preservative, a nutrititive culture medium, a polymer solution, or a combination of two or more thereof. FIG. 1C shows an image of an exemplary open pouch and FIG. 1D shows an image of an exemplary closed pouch containing tissue graft material according to this example. In some instances, the pouch may be closed around the tissue material with a first seam and one or more internal seams may be formed (i.e. positioned internal to one or more existing seams used to form the pouch) that reduce the internal volume of the cavity within which the tissue material is positioned, as shown in FIG. 1D.

The pouches described herein may be made from a variety of different permeable materials. The permeable material is generally a woven mesh-like material or an otherwise porous material. In some instances, more than one type of permeable material is used to form the pouch. In one aspect, the permeable material is biocompatible. In some instances, the permeable material may be a permanent, non-bioresorable material. Exemplary permanent materials include polypropylene, a polyethylene (PE), an expanded polytetrafluoroethylene (ePTFE), a woven cellulosic, a polyamide, or a polyester. Exemplary polyethylenes include low density PE (LDPE), medium density PE (MDPE), and high density PE (HDPE). Exemplary cellulosics included rayon, cotton, and paper. An exemplary polyamide is Nylon. An exemplary polyester is Mylar®. In other instances, the permeable material may be bioresorable such that, after implantation into a patient, the material will degrade over time and be absorbed by the patient's body. Exemplary bioresorable materials include polylactic acid (PLA), polyglycolic acid (PGA), copolymers of PLA and PGA (PLA/PGC copolymers), polycaprolactone, polydioxanone, human tissue derived mesh, gelatin, and certain silks. An exemplary human tissue derived mesh may be a material derived from collagen). Exemplary silks are those generated using silk fibers that are bioresorbable (see generally, for example, Vepari and Kaplan, Prog. Polym. Sci. 32(8-9):991-1007 (2007) and Silk Biomaterials for Tissue Engineering and Regenerative Medicine (ed. S. Kundu), Woodhead Publishing Ltd. 2014). Materials having similar properties to those listed above are also contemplated.

The permeable material of the pouches may be a woven, an expanded, a braided, and/or an extruded mesh material. The permeable material may have different degrees of porosity based on the manner of its manufacture. In some instances, the porosity of the permeable material of a pouch may be selected based on the size or nature of the tissue intended to be placed within the pouch. Generally, the pore size of the material (diameter of pores formed therein) would be smaller than the smallest portion of the tissue to be held therein. This configuration (pore size) allows fluid and moisture to pass through the permeable material and onto/into tissue graft material positioned within the pouch without disrupting or removing the tissue from the confinement of the pouch. For example, where the pouch is intended to contain particulate tissue, the pore size of the material will be smaller than the smallest particle size of the tissue particles. In another example, where the pouch is intended to contain larger pieces of tissue, the pore sizes may also be larger but still smaller than the smallest dimension of the tissue material to be confined therein. In some instances, the pore size of the permeable material may be in the range of about 50 micron to 10 mm and any size within this range that is sufficiently small to retain the desired tissue graft material within the pouch. For example, the pore size may be about 50 micron to 500 micron, or about 50 micron to about 1 mm, or about 100 micron to about 250 micron, or about 250 micron to about 750 micron, or about 500 micron to about 2 mm, or about 1 mm to about 5 mm. In some instances, the flexibility of the material is related to porosity of the material (number or size of pores) such that materials with increased porosity may be more flexible than materials with reduced porosity. The materials are generally medical grade. The materials may also be sufficiently durable to sustain sterilization using common sterilization techniques such as at least one of gamma irradiation, electron beam irradiation, ethylene oxide, supercritical carbon dioxide, and other common forms of sterilization and bioburden elimination. In some instances, the material does not adhere or stick to tissue. The lack of adherence to tissue by the permeable material may facilitate insertion or removal of the tissue graft from the pouch and positioning or manipulating the tissue graft when it is in contact with the permeable material (such as, for example, after opening the pouch).

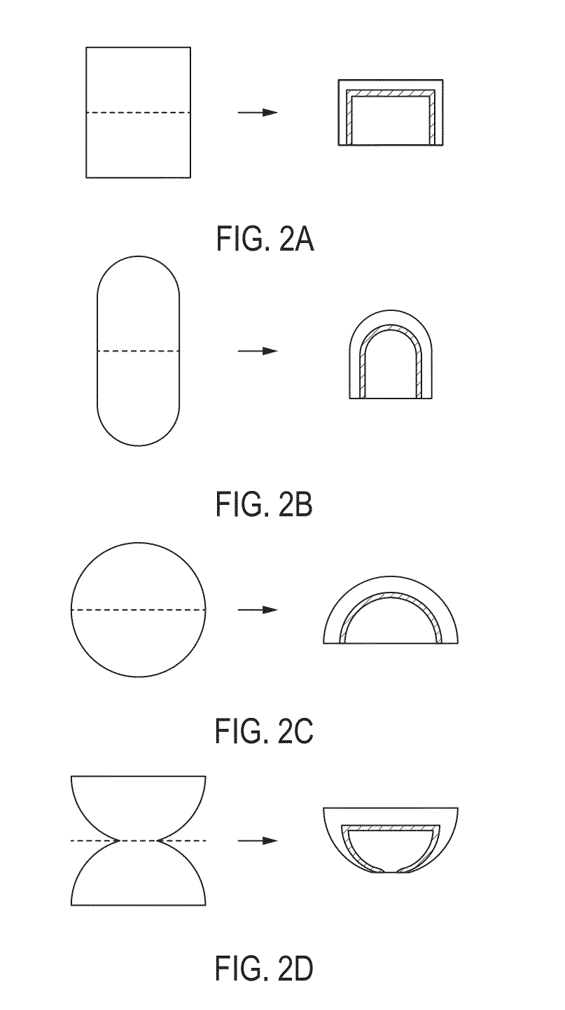

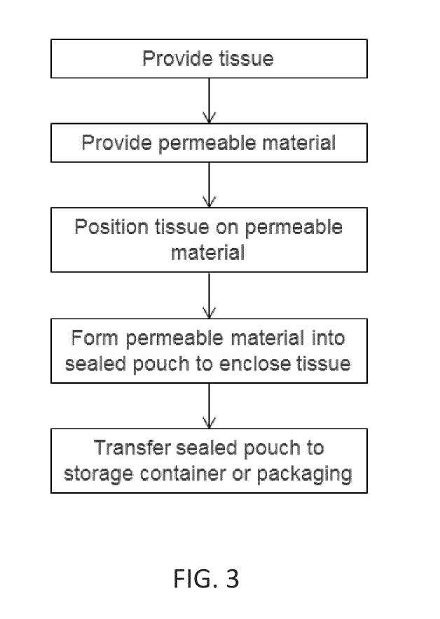

The pouches may be formed in various configurations suitable for containment of tissue graft material in different forms and volumes. For example, the exemplary pouch depicted in FIG. 1A, FIG. 1C, and FIG. 1D has a sqarish shape and a relatively flat geometry. Exemplary pouches having a relatively flat geometry are also shown in FIGS. 2A-2D. In some instances, the shape of the permeable material is such that, when folded, the desired geometry for the pouch is formed. In some examples, the permeable material may be folded once along a midline. For example, the shape of the permeable material on one side of the midline may be a mirror image the permeable material on the other side of the midline. In one example, the permeable material may be rectangular and form a sqarish shape when folded as shown in FIG. 2A. In another example, the permeable material may have a oval shape and form an elongated semi-circle shape when folded as shown in FIG. 2B. In another example, the permeable material may have a circular shape and form a semi-circular shape when folded as shown in FIG. 2C. In another example, the permeable material may have an hour-glass shape and form a semi-circular shape when folded as shown in FIG. 2D. In some instances, as described in FIG. 3, the sealed pouch may be formed around the tissue placed on the permeable material. For example, the tissue may be placed on a portion of the permeable material on one side of the midline, and then the permeable material may be folded along the midline to bring the edges of the permeable material into contact. Portions of the permeable material along the edges may then be sealed to enclose the tissue graft within a cavity formed between the two sides of the permeable material. In other examples, the permeable material may have a shape as shown in any of FIGS. 2A-2D and may be formed similar to as described in FIG. 1B such that an open pouch configuration is formed by not fully sealing all of the edges of the permeable material together after the material is folded (i.e. leaving an portion of the pouch where the peripheral edges of two adjacent portions or pieces of permeable material are not sealed together). The tissue graft material may then be inserted within the cavity formed between the sides of the folded permeable material. The pouch may then be sealed if desired.

In some instances, multiple pieces of permeable material may be used to form the pouches described herein. For example, the pouches shown in FIGS. 1A-1C and FIGS. 2A-2D may each be formed by adjoining (by seal or seam) two pieces of permeable material having the same, or generally the same, shape and size. An open pouch may be formed in this manner into which tissue graft material may be inserted. Alternatively, tissue graft material may be placed on one piece of permeable material and then overlaid with a second piece of permeable material. The edges of the two pieces of permeable material may then be adjoined (sealed) to enclose the tissue graft material.

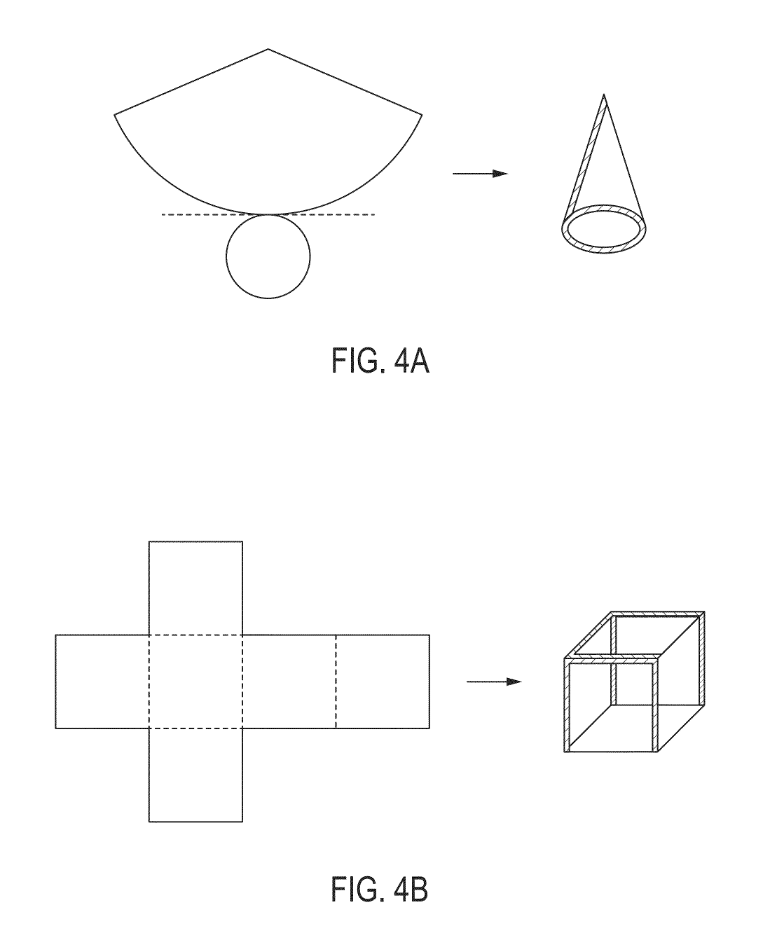

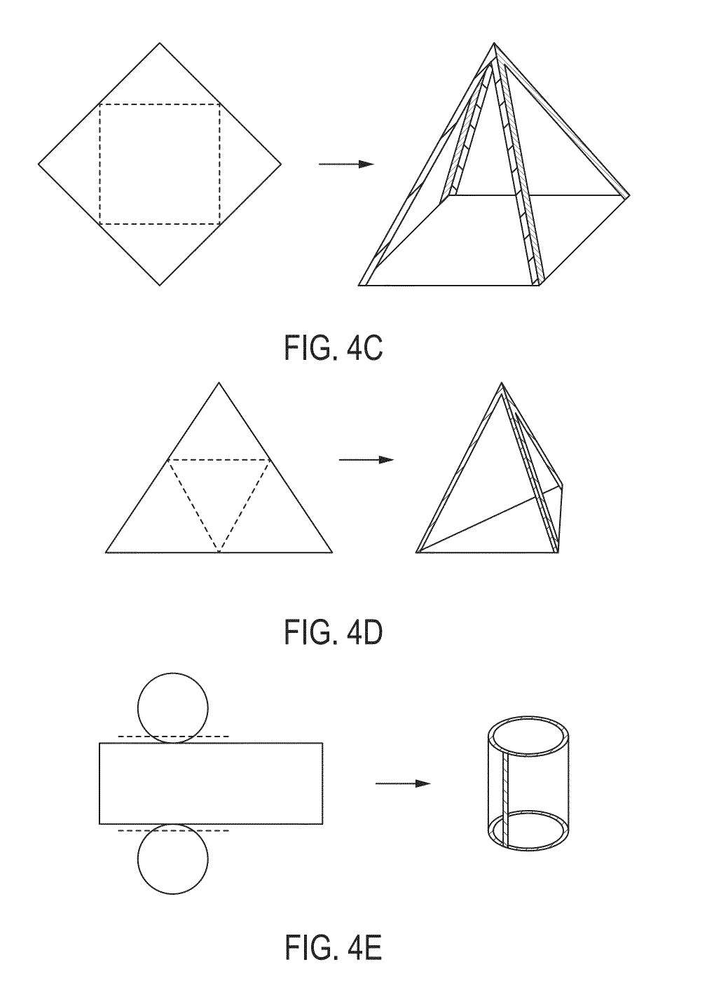

In certain instances, the geometry of the pouches may be a three-dimensional shape. For example, the pouch may be in the shape of a cone, a cube, a pyramid, or a tubular (cylindrical) shape. Such exemplary shapes are shown in FIGS. 4A-4E. In some instances, pouches having a three-dimensional shape may be formed from a single piece of permeable material that is folded along one or more fold lines to form the three-dimensional shape. In other instances, pouches having a three-dimensional shape may be formed from two or more pieces of permeable material that are adjoined (such as by seal or seam) to form the three-dimensional shape. In some instances, where two or more pieces of permable material are used to form the three-dimensional shape, one or more of the pieces may also be folded along one or more fold lines.

The pouches may have various sizes and dimensions. The size of the pouches can be tailored to contain tissue graft material in various forms and volumes. In some instances, the volume of the internal cavity of the pouch may be greater than the volume of the tissue graft to be inserted therein. For example, the internal cavity of the pouch may be some percent volume greater than the volume of the tissue graft to be inserted such as, for example, at least 5%, 10%, 15%, 20%, 25%, 30%, 35%, 40%, 45%, 50%, 55%, 60%, 65%, 70%, 75%, 80%, 85%, 90%, or 95% greater than the volume of the tissue to be inserted. For example, the volume of the internal cavity of the pouch may be between about 0.5 cc to about 300 cc. 1 cc, 1 cm³, and 1 mL are equivalent volumes.

To form the geometry of the pouch, seals or seams are formed along the edges or periphery of one or more piece of permeable material used to form the pouch to adjoin one part of a piece of material to another or to adjoin one piece of permeable material to one or more other pieces of material. The seams may be formed in the permeable material using various methods including, but not limited to, heat, pressure, heat and pressure. The sealing method used may be selected based on the type of permeable material. Table 1 sets forth exemplary sealing methods for different types of permeable materials and different types of bioresorbable materials. In some instances, seams may be formed in permanent materials as described in this disclosure using the sealing methods as set forth in the top half of Table 1. In some instances, seams may be formed in bioresorbable materials as described in this disclosure using the sealing methods as set forth in the bottom half of Table 1. In some instances, a heat, pressure, or heat and pressure may be used to form the seams (seals). For example, a bar sealer may be used to form the seams. In some instances, a laser beam may be used to form the seams (seals). In some instances, the seams (seals) may be formed by running an electric current through the adjacent pieces or portions of permeable material that are to be sealed together to form the seam (referred to as electric sealing). In some instances, the seams (seals) may be formed using ultrasonic welding.

In some instances, the material may be extruded into the desired shape. In some instances, the seams may be formed by sewing together portions of the permeable material, or different pieces of permeable material, that are adjacent in the closed pouch (final configuration). The thread used to sew the material together is medical grade and, where the permeable material is bioresorbable, the thread may also be bioresorable. In some instances, the seams (seals) may be formed using an adhesive to adhere together portions of the permeable material, or different pieces of permeable material, that are adjacent in the closed pouch (final configuration).

TABLE 1
Exemplary sealing methods for different types of permeable materials.

| Sealing methods: | | | | | | | | |
|---|---|---|---|---|---|---|---|---|
| | | | Electric sealing | | | | | |
| | | Heat + | (current through | | Extrude into | Ultrasonic | | Adhesives |
| Heat | Pressure | pressure | metal) | Laser | desired shape | Welding | Sewing | (gluing) |

Permanent materials

| | | | | | | | | |
|---|---|---|---|---|---|---|---|---|
| Polypropylene | x | | x | x | x | x | x | x |
| Polyethylene (PE) | | | x | x | x | x | x | x |
| Low density (LDPE) | x | | x | x | x | x | x | x |
| Medium density (MDPE) | x | | x | x | x | x | x | x |
| High density (HDPE) | x | | x | x | x | x | x | x |
| expanded polytetraflluoroethylene | x | | x | x | x | x | x | x |
| (ePTFE) | | | | | | | | |
| woven cellulosics (such as rayon, | | | x | x | x | x | x | x |
| cotton, paper) | | | | | | | | |
| Polyamides (such as Nylon and | x | x | x | x | x | x | x | x |
| others) | | | | | | | | |
| Polyesters (such as Mylar ® and | x | x | x | x | x | x | x | x |
| others) | | | | | | | | |

Bioresorbable materials

| | | | | | | | | | |
|---|---|---|---|---|---|---|---|---|---|
| Polylactic acid (PLA) | x | | x | x | | x | x | x¹ | x |
| Polyglycolic acid (PGA) | x | | x | x | | x | x | x¹ | x |
| Copolymers of PLA & PGA | x | | x | x | | x | x | x¹ | x |
| (PLA/PGA copolymers) | | | | | | | | | |
| Polycaprolactone | x | | x | | | x | x | x¹ | x |
| Polydioxanone | | | x | | | x | x | x¹ | x |
| Human Tissue Derived mesh (such | | x | | | x | | | x¹ | x |
| as material derived from collagen) | | | | | | | | | |
| Gelatin | | | | | | x | | x¹ | x |
| Silk | | | | | | | x | x¹ | x |

x¹: Can be sewn with resorbable thread.

In some instances, the seals or seams can have a sufficiently weak tensile strength that a user may easily separate the joined seams to open the sealed pouch manually or using forcepts but sufficiently strong tensile strength such that the seams retain integrity during normal handling of the pouch. For example, a weak tensile strength may be pull strength of at least about 1 pounds-force but less than about 3.8 pounds-force pull strength. For example, the weak tensile strength may be pull strength of about 1 pounds-force, 1.25 pounds-force, 1.5 pounds-force, 1.75 pounds-force, 2 pounds-force, 2.25 pounds-force, 2.5 pounds-force, 2.75 pounds-force, 3 pounds-force, 3.25 pounds-force. 3.5 pounds-force, or a tensile strength within 20% of any of these tensile strengths. In some instances, a weak tensile strength may be pull strength of about 1 pounds-force. In some instances, a seam having a pull strength below 1 pounds-force may not have sufficient integrity to retain its integrity during normal handling of the pouch. In some cases, the seals or seams may have sufficient tensile strength to generally prevent separation of the joined seams by manual separation such that opening the pouch would require the permeable material of the pouch to be cut. For example, a stronger tensile strength may be about 4-5 pounds-force pull strength. In some instances, generating a pull strength greater than about 5 pounds-force using heat, pressure, heat and pressure, electric sealing, or laser may result in damage to the permeable material of the pouch.

In one example, as described below, the pouch may be made from a polypropylene mesh material having a pore size of 210 micron. In some instances, a heat sealer may be used to form the seams in the permeable material. In one example, the settings used for the heat sealer to form seams having a pull strength of about 4-5 pounds-force are about 375° F. seal temperature, 220° F. release temperature, 4 sec seal time, and 85 psi seal pressure.

In some instances, the pouches described herein may be part of an implantable tissue graft product comprising a sealed pouch as described herein containing tissue positioned within the cavity of the pouch (for example, forming a tissue capsule). For example, the use of bioresorbable permeable materials as described in this disclosure to manufacture the pouch would permit the implantation into a defect site of the tissue-containing pouch without the need to remove the tissue from the pouch prior to implantation. One advantage to implanting the tissue-containing pouch at a defect site is that, for particulate tissue or grafts comprising multiple small pieces of tissue, containment in the pouch prevents the tissue from traveling away from the defect site once implanted.

### Method of Use

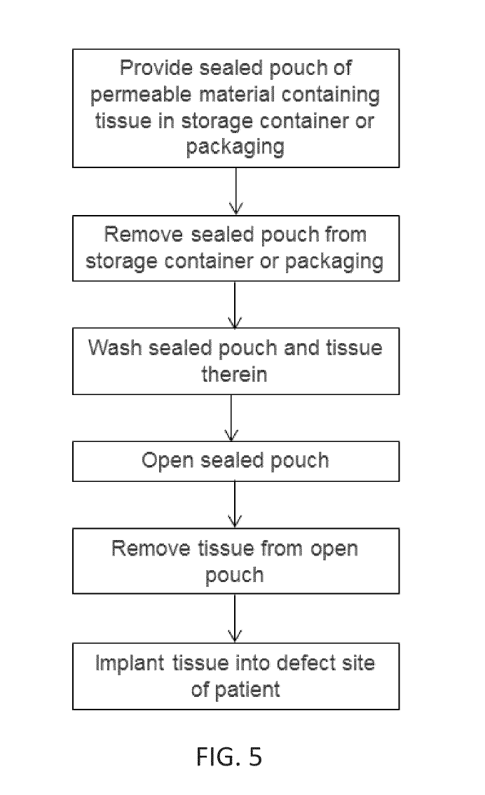

The pouches described herein may be used to treat a subject in need of a tissue graft. For example, as depicted in FIG. 5, a sealed pouch made of permeable material and containing tissue for use in a tissue graft may be provided in a storage container or other packaging such as plastic or glass vials or bags. The storage container or other packaging may contain a wetting agent. In some instances, the pouch containing the tissue may be frozen and requires being thawed prior to use. When ready for use, the pouch may be removed from the storage container or other packaging. In some instances, the pouch may be manipulated by the user. For example, the user may wash the pouch containing the tissue with one or more wash solutions. To access the tissue directly, the sealed pouch may be opened by manually separating the seams of the pouch such as by pulling or peeling the permeable material on one side of the seam away from the permeable material on the other side of the seam. For example, one or more of the seams may be separated to open the pouch and provide access to the internal cavity and the tissue therein. The tissue may then be removed from open pouch. In some instances, all of the seams or seals in the permeable material that form the pouch may be separated. For example, the permable material of the pouch may be laid flat on a work surface with the tissue positioned on its surface, which may create a useful platform for the tissue. In some instances, the tissue may be further manipulated, such as, for example, by washing, mixing, or combining with other reagents. The tissue may then be implanted into a defect site of a subject.

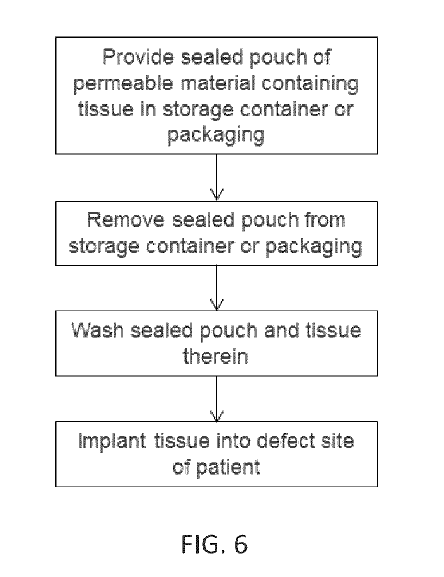

In some instances, as depicted in FIG. 6, the method of treatment may comprise implanting an sealed pouch into a subject in need of a tissue graft. The method is substantially similar to that described above for FIG. 5 except that the sealed pouch is not opened. Rather, the pouch, which contains the tissue, is implanted into a defect site of a subject. This can be useful where the tissue to be implanted is particulate tissue or a collection of small pieces of tissue. Containment of the tissue in the pouch may retain the implanted tissue at the defect site.

### Unsealed Pouches

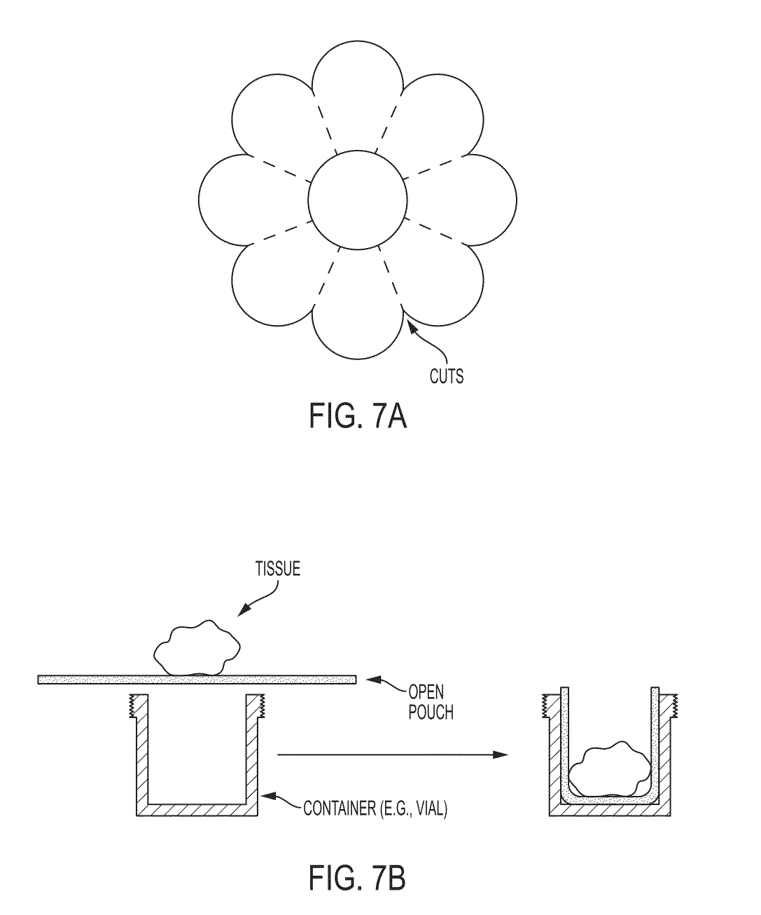

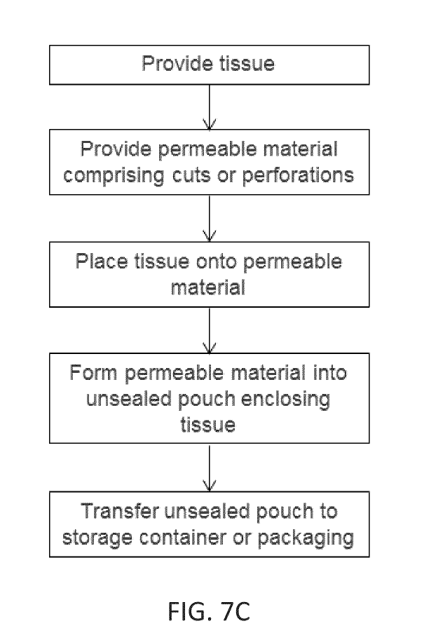

In some aspects, the pouches may be unsealed pouches. An exemplary unsealed pouch is shown in FIG. 7A-7B. In some instances, as described in FIG. 7C, an unsealed pouch is formed from permeable material and is configured to fold into a pouch shape. In contrast to a sealed pouch, unsealed pouches may not have seams or seals formed therein that adjoin portions of the permeable material to each other to form a fixed pouch configuration. Rather, the permeable material may have perforations or cuts formed therein, as shown in FIG. 7A, that permit portions of the permeable material to fold towards each other to form an unsealed pouch. The number of perforations or cuts in the permeable material may vary based on several factors including, for example, the size of the permeable material, the shape of the tissue graft to be enclosed therein, and the size or shape of the storage container or packaging into which it will be placed. The permeable material may have 1, 2, 3, 4, 5, 6, 7, 8, 9, 10, 11, 12, 13, 14, 15, or some other number of perforations or cuts. In some instances, a tissue graft may be placed on the permeable material and the material folded around the tissue graft to enclose the tissue graft within the unsealed pouch. The tissue graft within in the unsealed pouch may be transported from one work surface to another or inserted or removed from a storage container or packaging as shown in FIG. 7B. In some instances, a unsealed pouch may be placed into a storage container or packaging such as, for example, one or more plastic or glass vials or bags. In some instances, the storage container or packaging may be filled with a wetting solution such as, for example, sterile water, saline, a cryoprotective solution, a preservative, a nutrititive culture medium, a polymer solution, or a combination of two or more thereof.

As described in FIG. 7C, in some instances, the unsealed pouch may be removed from a storage container or packaging, and the permeable material may be opened and laid flat with the tissue positioned thereon. The tissue may then be washed or otherwise manipulated prior to being implanted into a defect site of a patient.

### EXAMPLES

A study was performed to determine optimal heat sealer parameters to form seams in polypropylene 210 micron mesh material (SEFAR PROPYLTEX 05-210/32). This material is an exemplary permeable material for use in manufacturing a pouch according to this disclosure. The porosity (pore size) of this material in 210 μm. The heat sealer used in this study to form seams in the material was a Model 730 Validatable Medical Sealer (Accu-Seal Corp., San Marcos, Calif.). The tensile strength of the seals was tested with an ADMET 2600 tensile tester and an Interface Load Cell (model SM-100, S/N D73997).

Initial testing was conducted to determine an appropriate range for the heat sealer parameters. The foil-on-foil heat sealer settings shown in Table 2 were used as a starting point.

TABLE 2
Foil-on-Foil Program Settings

| | | |
|---|---|---|
| Seal Temperature | 370° | F. |
| Release Temperature | 220° | F. |
| Seal Time | 5.0 | sec. |
| Seal Pressure | 85 | psi |
| Voltage | 115 | V |
| Temperature Band Alarm | ±15° | F. |
| Pressure Band Alarm | ±10 | psi |
| Voltage Band Alarm | ±15 | VAC |
| Cold Seal Alarm | 15 | sec. |

Using modifications of the above settings, 6 inch strips of the material were cut and sealed according to each setting shown in Table 3. The strips were then pulled apart using the ADMET 2600 tensile tester to determine the peak pull strength of the sample. The target pull strength for this experiment was 4-5 pound-force (lbf). This pull strength is sufficient that the seam will not separate from routine handling of a pouch made using this material and seams of this strength. This pull strength is also sufficient that the material of a pouch made using this material and seams of this strength would need to be cut to open.

TABLE 3
Preliminary Testing Results

| | Parameters Compared to Table 2 | | | | Results | | |
|---|---|---|---|---|---|---|---|
| | | Δ | | | | Load | Load |
| | Δ Seal | Release | Δ Seal | Δ | | Strength | Strength |
| | Temp. | Temp. | Time | Pressure | Seal | Trial 1 | Trial 2 |
| Run | (° F.) | (° F.) | (s) | (psi) | Formed | (lbs.) | (lbs.) |
| 1 | −20 | — | — | — | Yes | 0.4 | 0.9 |
| 2 | −10 | — | — | — | Yes | 1.7 | 1.5 |
| 3 | +10 | — | — | — | Yes | 13.4 | 15.3 |
| 4 | +20 | — | — | — | Yes | 13.5 | 12.7 |
| 5 | — | −20 | — | — | Yes | 4.3 | 5.0 |
| 6 | — | −10 | — | — | Yes | 2.1 | 3.3 |
| 7 | — | +10 | — | — | Yes | 3.2 | 2.4 |
| 8 | — | — | −2 | — | Yes | 1.5 | 1.9 |
| 9 | — | — | −1 | — | Yes | 2.6 | 3.6 |
| 10 | — | — | +1 | — | Yes | 3.3 | 4.2 |
| 11 | — | — | +2 | — | Yes | 4.7 | 5.3 |
| 12 | — | — | — | −5 | Yes | 2.8 | 2.7 |
| 13 | — | — | — | +5 | Yes | 3.1 | 3.8 |
| 14 | −20 | +10 | −2 | −5 | No | — | — |
| 15 | −20 | −20 | +2 | — | Yes | 0.5 | 1.2 |
| 16 | −20 | −20 | −2 | — | Yes | 0.4 | 0.2 |
| 17 | −20 | +10 | +2 | — | Yes | 0.7 | 0.2 |
| 18 | −20 | +10 | −2 | — | Yes | 0.1 | 0.1 |
| 19 | +20 | −20 | +2 | — | Yes | 6.5 | 8.4 |
| 20 | +20 | −20 | −2 | — | Yes | 10.2 | 5.5 |
| 21 | +20 | +10 | +2 | — | Yes | 4.9 | 5.8 |
| 22 | +20 | +10 | −2 | — | Yes | 14.2 | 11.9 |
| 23 | +10 | +10 | +2 | — | Yes | 13.1 | 9.1 |
| 24 | +10 | +10 | −2 | — | Yes | 12.0 | 11.3 |
| 25 | +10 | −10 | +2 | — | Yes | 9.9 | 12.2 |
| 26 | +10 | −10 | −2 | — | Yes | 11.0 | 10.7 |
| 27 | −10 | +10 | +2 | — | Yes | 4.0 | 2.0 |
| 28 | −10 | +10 | −2 | — | Yes | 0.4 | 0.5 |
| 29 | −10 | −10 | +2 | — | Yes | 1.7 | 3.4 |
| 30 | −10 | −10 | −2 | — | Yes | 0.4 | 0.6 |
| 31 | +10 | −10 | −1 | +5 | Yes | 11.3 | 9.9 |
| 32 | +10 | −10 | +1 | −5 | Yes | 15.0 | 11.9 |
| 33 | +20 | −20 | +2 | +5 | Yes | 10.9 | 13.2 |

A review of the data from this initial testing resulted in the preliminary center point for the design of experiment (DOE) shown in Table 4. The intention of the DOE was to gain information on the main effects and interactions of each variable to determine the optimal set points.

TABLE 4
Parameter Comparison

| | Parameter Values | | |
|---|---|---|---|
| | Preliminary | DOE | |
| Parameter | Center Point | Minimum | Maximum |
| Seal Temperature (° F.) | 380 | 370 | 390 |
| Release Temperature (° F.) | 215 | 200 | 230 |
| Seal Time (seconds) | 5.0 | 3.0 | 7.0 |
| Seal Pressure (psi) | 85 | 80 | 90 |

Minitab Statistical Analysis Software was used to determine which variables and interactions showed a statistically significant (α=0.05) effect. Temperature and time significantly affect the strength of the seal. The interaction between time and pressure also has a significant impact on the pull strength but only weakly.

The Minitab software was also used to predict pull strengths based on the heat sealer settings shown in Table 5. Release temperature and pressure were held constant, while temperature and time were adjusted within the ranges set by the DOE. The optimal setting was chosen as the setting with a predicted pull strength between 4 and 5 pounds-force.

TABLE 5
Predictive Pull Strengths

| | Release | | | | Confi- | Confi- |
|---|---|---|---|---|---|---|
| Temper- | Temper- | | | Pull | dence | dence |
| ature | ature | Time | Pressure | strength | Interval - | Interval - |
| (° F.) | (° F.) | (s) | (psi) | (lbs) | Low | High |
| 370 | 220 | 3 | 85 | 1.672 | 0.277 | 3.068 |
| 370 | 220 | 4 | 85 | 3.169 | 2.065 | 4.272 |
| 370 | 220 | 5 | 85 | 4.665 | 3.678 | 5.652 |
| 370 | 220 | 6 | 85 | 6.161 | 5.058 | 7.264 |
| 370 | 220 | 7 | 85 | 7.657 | 6.262 | 9.053 |
| 375 | 220 | 3 | 85 | 3.494 | 2.39 | 4.597 |
| 375 | 220 | 4 | 85 | 4.796 | 3.924 | 5.668 |
| 375 | 220 | 5 | 85 | 6.099 | 5.319 | 6.879 |
| 375 | 220 | 6 | 85 | 7.401 | 6.529 | 8.273 |
| 375 | 220 | 7 | 85 | 8.704 | 7.6 | 9.807 |
| 380 | 220 | 3 | 85 | 5.315 | 4.328 | 6.302 |
| 380 | 220 | 4 | 85 | 6.424 | 5.644 | 7.204 |
| 380 | 220 | 5 | 85 | 7.532 | 6.835 | 8.23 |
| 380 | 220 | 6 | 85 | 8.641 | 7.861 | 9.421 |
| 380 | 220 | 7 | 85 | 9.75 | 8.763 | 10.737 |
| 385 | 220 | 3 | 85 | 7.136 | 6.033 | 8.239 |
| 385 | 220 | 4 | 85 | 8.051 | 7.179 | 8.923 |
| 385 | 220 | 5 | 85 | 8.966 | 8.186 | 9.746 |
| 385 | 220 | 6 | 85 | 9.881 | 9.009 | 10.753 |
| 385 | 220 | 7 | 85 | 10.796 | 9.693 | 11.9 |
| 390 | 220 | 3 | 85 | 8.957 | 7.562 | 10.353 |
| 390 | 220 | 4 | 85 | 9.679 | 8.575 | 10.782 |
| 390 | 220 | 5 | 85 | 10.4 | 9.413 | 11.387 |
| 390 | 220 | 6 | 85 | 11.121 | 10.018 | 12.225 |
| 390 | 220 | 7 | 85 | 11.843 | 10.447 | 13.238 |

Study Conclusion: Temperature, time, and time and pressure have a significant effect on the pull strength of the mesh material. The DOE results indicate that the settings set forth in Table 6 are optimal for this material to generate seams having a pull strength of 4-5 pounds-force.

TABLE 6
Optimal Parameters for Material & Target Pull Strength

| Parameter | Optimal |
|---|---|
| Seal Temperature (° F.) | 375 |
| Release Temperature (° F.) | 220 |
| Seal Time (seconds) | 4 |
| Seal Pressure (psi) | 85 |

All patents, patent publications, patent applications, journal articles, books, technical references, and the like discussed in the instant disclosure are incorporated herein by reference in their entirety for all purposes.

It is to be understood that the figures and descriptions of the invention have been simplified to illustrate elements that are relevant for a clear understanding of the invention. It should be appreciated that the figures are presented for illustrative purposes and not as construction drawings. Omitted details and modifications or alternative embodiments are within the purview of persons of ordinary skill in the art.

It can be appreciated that, in certain aspects of the invention, a single component may be replaced by multiple components, and multiple components may be replaced by a single component, to provide an element or structure or to perform a given function or functions. Except where such substitution would not be operative to practice certain embodiments of the invention, such substitution is considered within the scope of the invention.

The examples presented herein are intended to illustrate potential and specific implementations of the invention. It can be appreciated that the examples are intended primarily for purposes of illustration of the invention for those skilled in the art. There may be variations to these diagrams or the operations described herein without departing from the spirit of the invention. For instance, in certain cases, method steps or operations may be performed or executed in differing order, or operations may be added, deleted or modified.

Different arrangements of the components depicted in the drawings or described above, as well as components and steps not shown or described are possible. Similarly, some features and sub-combinations are useful and may be employed without reference to other features and sub-combinations. Embodiments of the invention have been described for illustrative and not restrictive purposes, and alternative embodiments will become apparent to readers of this patent. Accordingly, the present invention is not limited to the embodiments described above or depicted in the drawings, and various embodiments and modifications can be made without departing from the scope of the claims below.

What is claimed is:

1. A pouch for tissue containment comprising at least one piece of permeable material having a geometrical shape and a cavity enclosed by the permeable material, the permeable material comprising seams formed therein to delineate the cavity.

2. The pouch of claim 1, wherein the permeable material comprises a biocompatible material.

3. The pouch of claim 1, wherein the permeable material is porous.

4. The pouch of claim 1, wherein the permeable material is a woven material.

5. The pouch of claim 1, wherein the permeable material comprises a bioresorbable material.

6. The pouch of claim 1, wherein the cavity has a volume comprising about 0.5 cc to about 300 cc.

7. The pouch of claim 1, wherein the geometrical shape of the permeable material is a flat geometrical shape, a cone, a cube, a pyramid, or a cylindrical shape.

8. The pouch of claim 1, wherein the seams in the permeable material have a pull strength of about 4-5 pounds-force.

9. The pouch of claim 1, wherein the seams in the permeable material have a pull strength of about 1 pounds-force.

10. The pouch of claim 1, wherein the permeable material comprises pores having a diameter in the range of about 50 micron to about 10 mm.

11. The pouch of claim 1, wherein the permeable material comprises pores having a diameter in the range of about 150 micron to about 250 micron.

12. The pouch of claim 1, further comprising tissue graft material positioned within the cavity.

13. The pouch of claim 1, comprising a plurality of pieces of the permeable material, wherein the plurality of pieces are connected to each other via seams formed therein to delineate the cavity.

14. The pouch of claim 12, wherein the pouch comprises more than one type of permeable material.

15. A method of making a pouch for tissue containment, the method comprising:
(a) providing a permeable material;
(b) folding the permeable material into a geometrical configuration by bringing at least one edge or surface of the permeable material into contact with at least one other edge or surface of the permeable material, wherein the three-dimensional configuration comprises an internal cavity formed therein; and
(c) forming a seal between the at least one edge or surface of the permeable material and the at least one other edge or surface of the permeable material.

16. The method of claim 15, further comprising inserting tissue within the internal cavity.

17. The method of claim 16, further comprising forming a seal in at least one additional edge or surface of the permeable material to enclose the inserted tissue.

18. An unsealed pouch for tissue containment made by the method of claim 15.

19. A method of making a pouch for tissue containment, the method comprising:
(a) providing a plurality of pieces of permeable material;
(b) positioning the pieces of permeable material into a geometrical configuration by bringing at least one edge or surface of a first piece of permeable material into contact with at least one other edge or surface of a second piece of permeable material, wherein the geometrical configuration comprises an internal cavity formed therein; and
(c) forming a seal between the at least one edge or surface of the first permeable material and the at least one edge or surface of the second permeable material.

20. The method of claim 19, further comprising inserting tissue within the internal cavity.

21. The method of claim 20, further comprising forming a seal in at least one additional edge or surface of the permeable material to enclose the inserted tissue.

22. A method of treating a patient having a tissue defect site in need of a tissue graft, the method comprising:
(a) providing the pouch of claim 1;
(b) opening the pouch to expose the tissue contained therein by separating at least some of the seams in the permeable material;
(c) extracting the tissue from the cavity of the pouch; and
(d) implanting the tissue into the tissue defect site of the patient.

23. The method of claim 22, further comprising manipulating the pouch prior to opening the pouch.

24. The method of claim 22, wherein the pouch is washed with a wash solution.

25. The method of claim 22, wherein the pouch is soaked in a wetting agent to rehydrate the tissue contained therein.

26. The method of claim 22, wherein all of the seams in the permeable material are separated.

27. The method of claim 22, further comprising removing the pouch from a storage container or packaging before inserting the pouch into the patient.

28. A method of treating a patient with a permeable pouch containing tissue graft material, the method comprising inserting the pouch into a wound site of a patient in need of a tissue graft.

29. The method of claim 28, wherein the pouch is washed with a wash solution.

30. The method of claim 28, wherein the pouch is soaked in a wetting agent to rehydrate the tissue contained therein.

31. The method of claim 28, further comprising removing the pouch from a storage container or packaging before inserting the pouch into the patient.

32. A method of treating a patient having a tissue defect site in need of a tissue graft, the method comprising:
(a) providing the unsealed pouch of claim 18, wherein the unsealed pouch contains tissue;
(b) unfolding the permeable material of the unsealed pouch to expose the tissue; and
(c) implanting the tissue into the tissue defect site of the patient.

33. The method of claim 32, further comprising manipulating the unsealed pouch prior to unfolding the pouch.

34. The method of claim 32, wherein the unsealed pouch is washed with a wash solution.

35. The method of claim 32, wherein the pouch is soaked in a wetting agent to rehydrate the tissue contained therein.
